(19) United States
(12) Patent Application Publication
(10) Pub. No.: US 2002/0103404 A1
(43) Pub. Date: Aug. 1, 2002

(54) Process for the nuclear chlorination of meta-xylene

(76) Inventors: Franz-Josef Mais, Dusseldorf (DE); Alexander Klausener, Pulheim (DE)

Correspondence Address: BAYER CORPORATION, PATENT DEPARTMENT, 100 BAYER ROAD, PITTSBURGH, PA, 15205, US

(21) Appl. No.: 09/928,540

(22) Filed: Aug. 13, 2001

(30) Foreign Application Priority Data
Aug. 14, 2000 (DE) 10039717.4

(51) Int. Cl. C07C 17/013

(52) U.S. Cl. 570/234

(57) ABSTRACT

This invention relates to a process for the nuclear chlorination of m-xylene using elemental chlorine in the presence of a Friedel-Crafts catalyst and a co-catalyst, where the co-catalysts used are benzo-fused thiazepines, or thiazocines.

### BACKGROUND OF THE INVENTION

The present invention relates to a process for the nuclear chlorination of m-xylene to give a mixture of 4-chloro-1,2-dimethylbenzene and 2-chloro-1,3-dimethylbenzene using elemental chlorine in the presence of a catalyst and a co-catalyst.

Mononuclear chlorinated o-xylenes are valuable intermediates for preparing agricultural and pharmaceutical active compounds and for preparing polymer precursors.

The nuclear chlorination of m-xylene by elemental chorine is known. For example, J. Org. Chem 41, 1976, 3580-3582, describes the fact that in the monochlorination of m-xylene in the presence of customary Lewis acid catalysts, for example, iodine, FeCl₃, or SbCl₃, an isomeric ratio of 4-chloro-1,3-dimethylbenzene to 2-chloro-1,3-dimethylbenzene of approximately 3.0:1 is obtained. 4-Chloro-1,3-dimethylbenzene is the more valuable isomer, so that a number of processes have been described for increasing the proportion of 4-chloro-1,3-dimethylbenzene.

U.S. Pat. No. 4,190,609 discloses a process for the nuclear chlorination of m-xylene by elemental chlorine, the procedure being carried out in the presence of Lewis acids as catalysts and defined substituted thianthrenes as co-catalysts. Although the ratio of 4-chloro-1,3-dimethylbenzene to 2-chloro-1,3-dimethylbenzene can by this method be increased to 3.62:1, a disadvantage in the use of thianthrenes is that compounds of this class act in a similar manner to the corresponding dioxins, that is to say they are toxic.

EP 126,669 A1 describes catalytic systems that consist of Lewis acids and N-substituted phenothiazines for the nuclear chlorination of aromatics. No example of the nuclear chlorination of m-xylene is given.

A fundamentally different process is the nuclear chlorination of m-xylene by chlorine in the presence of zeolites as catalysts. J. Catal. 150, 1994, 430-433 discloses that, when using a KL-zeolite in 1,2-dichloroethane solvent, a ratio of 4-chloro-1,3-dimethylbenzene to 2-chloro-1,3-dimethylbenzene of up to 6.5:1 can be achieved, but in this case over 61% of the m-xylene used is not converted. A further disadvantage of carrying out the process in the presence of zeolites is the use of a solvent and the heterogeneous catalyst, as a result of which, during the work-up of the reaction mixture, additional distillation steps and filtration steps become necessary.

An object of the present invention was to provide a process for the nuclear chlorination of m-xylene using a simply handled catalyst system, in which as high a possible a ratio of 4-chloro-1,3-dimethylbenzene to 2-chloro-1,3-dimethylbenzene is to be achieved.

This object is achieved in a surprisingly simple manner by using benzo-fused thiazepines or thiazocines as co-catalysts.

### SUMMARY OF THE INVENTION

The invention therefore relates to a process for the nuclear chlorination of m-xylene comprising chlorinating m-xylene with elemental chlorine in the presence of Friedel-Crafts catalysts and benzo-fused thiazepines or thiazocines as co-catalysts.

### DETAILED DESCRIPTION OF THE INVENTION

Suitable Friedel-Crafts catalysts for the inventive process are known. Examples that may be mentioned are: antimony chlorides, antimony oxides, aluminum chloride, iron(II) chloride, iron(III) chloride, tellurium chlorides, lead chlorides, molybdenum chlorides, tin chlorides, tungsten chlorides, titanium chlorides, zinc chlorides, boron trichloride, and boron trifluoride.

Elements and element compounds that form a Friedel-Crafts catalyst, that is to say a Lewis acid, during the chlorination can also be used (precursors of Friedel-Crafts catalysts), for example, the metals or semi-metals antimony, iron, lead, tin, zinc, molybdenum, tellurium, or aluminum or their oxides, sulfides, carbonyls, or salts, for example, carbonates. Examples of element compounds coming into consideration are antimony oxides, iron oxides, iron sulfides, lead sulfides, tin sulfides, zinc sulfides, iron carbonyls, molybdenum carbonyls, and boron phosphate. Instead of the chlorides mentioned, the corresponding fluorides, bromides, and if appropriate iodides of the said elements can also be used.

Preference is given in the inventive process to antimony chlorides, iron, iron oxides, iron sulfides, iron carbonyls, and iron(III) chloride as Friedel-Crafts catalyst. Particular preference is given to iron(III) chloride.

Friedel-Crafts catalysts and/or their precursors can be used individually or as any mixtures with one another.

The amount of the Friedel-Crafts catalyst or its precursor can be varied within broad limits. Thus, frequently, catalyst activity can be observed even at an addition of 0.0005% by weight. On the other hand, 5% by weight or more of the Friedel-Crafts catalyst can also be added, but such high amounts generally offer no advantages and may even be accompanied by disadvantages during work-up. Usually, the Friedel-Crafts catalyst is used in an amount of 0.001 to 1.0% by weight, preferably 0.005 to 0.5% by weight. All these figures are based on the amount of the m-xylene used.

In the inventive process the co-catalysts used are thiazepines or thiazocines. Processes for preparing such compounds are known and are described, for example, in U.S. Pat. No. 4,948,886.

For example, the co-catalysts used can be benzothiazepines of the formulas

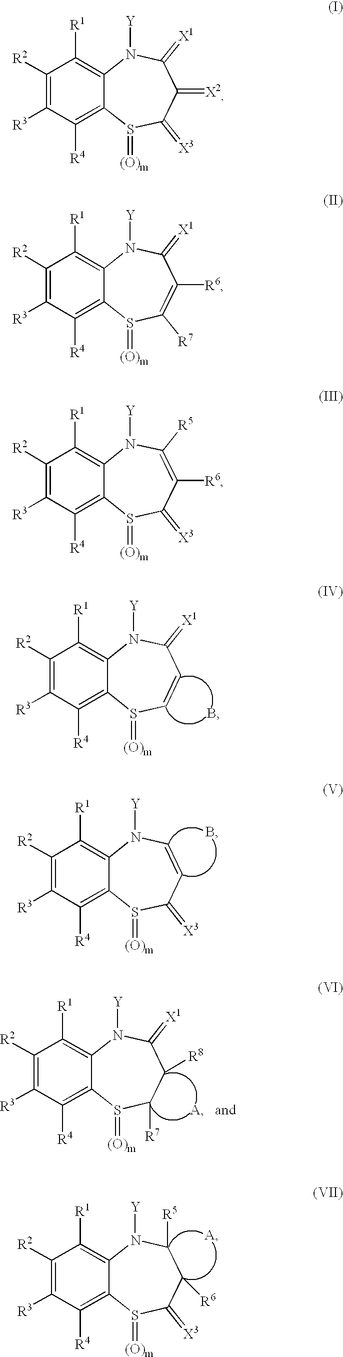

where

R¹, R², R³, and R⁴ are identical or different and represent hydrogen, hydroxyl, amino, cyano, halogen, nitro, nitroso, sulfonyl, sulfoxyl, tosyl, mercapto, carboxyl, carboxyamide, carbalkoxy, dithiocarboxyl, thiocarboxcyamide, dithiocarbalkoxy, or unsubstituted or substituted alkyl, aryl, heteroaryl, alkoxy, aryloxy, heteroaryloxy, acyloxy, alkylthio, arylthio, heteroarylthio, acylthio, acyl, thioacyl, or acylamino and, in addition, can together form one or more saturated or unsaturated, unsubstituted or substituted isocyclic or heterocyclic carbon rings having up to 8 carbon atoms.

R⁵, R⁶, R⁷, and R⁸ are identical or different and have the meanings of R¹ to R⁴ except that they cannot together form rings,

Y denotes hydrogen, unsubstituted or substituted alkyl, aryl, heteroaryl, acyl, thioacyl, acyloxy, arylamino, or acylamino,

X¹, X², or X³ independently of one another each denotes one of the following groups

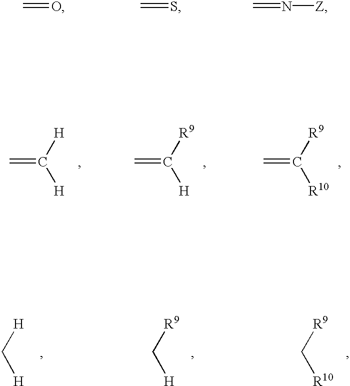

where

R⁹ and R¹⁰ are identical or different and have the meanings of R⁵ to R⁸, and

Z has the meaning of Y except that Z cannot be identical to H,

A denotes the anellation of an unsubstituted or substituted saturated isocyclic or heterocyclic ring having up to 8 carbon atoms,

B denotes the anellation of an unsubstituted or substituted unsaturated isocyclic or heterocyclic ring having up to 8 carbon atoms, and

m denotes 0 or 1.

In addition, the co-catalysts can be, for example, compounds of the formula

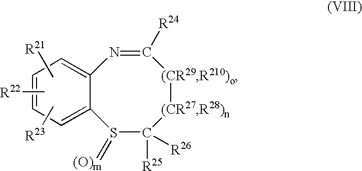

where

R²¹ and R²² independently of one another denote hydrogen, hydroxyl, amino, cyano, halogen, nitro, carboxyl, halogenocarbonyl, carboxyamide, alkoxycarbonyl, alkyl, aryl, alkoxy, aryloxy, acyloxy, alkylthio, arylthio, acylthio, acyl, thioacyl, or acylamino,

R²³ represents hydrogen or chlorine and, in addition with an adjacently ring-substituted radical R²¹ or R²² and together with the substituted carbon atoms, can form an anellated saturated, unsaturated, or aromatic isocyclic or heterocyclic ring having 5 to 8 ring atoms,

R²⁴ denotes hydrogen, alkyl, aryl, halogen, alkylthio, arylthio, alkoxy, aryloxy, amino, hydrazino, alkylhydrazino, or phenylhydrazino,

m, n, and o independently of one another can have the value 0 or 1, but n and o cannot simultaneously have the value 0,

R²⁵, R²⁷, and R²⁹ independently of one another denote hydrogen, alkyl, alkoxy, phenyl, acyloxy, cyano, halogen, carboxyl, alkoxycarbonyl, phenoxy, or acyl, where R²⁵ and R²⁷ or R²⁷ and R²⁹, together with the substituted carbon atoms, can form a saturated, unsaturated, or aromatic isocyclic or heterocyclic ring having 5 to 8 ring atoms,

R²⁶, R²⁸, and R²¹⁰ independently of one another denote hydrogen, alkyl, or halogen, where R²⁶ and R²⁸ or R²⁸ and R²¹⁰ can together form a double bond, where, in addition, R²⁵ and R²⁶ can together designate double-bonded oxygen, sulfur, or R²¹¹-substituted nitrogen, where R²¹¹ denotes alkyl, aryl, acyl, alkylamino, or arylamino.

In addition, the co-catalysts used can be, for example, compounds of the formula

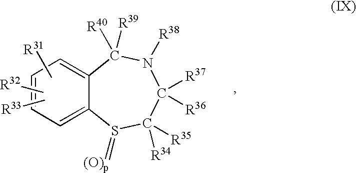

where

R³¹ and R³² independently of one another denote hydrogen, hydroxyl, amino, cyano, halogen, nitro, C₁-C₈-alkyl, phenyl that is unsubstituted or substituted by R³¹ and R³² (except for repeated substitution by R³¹- and R³²-substituted phenyl), C₁-C₈-alkoxy, phenoxy, C₁-C₈-acyloxy, C₁-C₈-acyl, or C₁-C₈-alkoxycarbonyl,

R³³ represents hydrogen or chlorine and furthermore with one of the radicals R³¹ or R³² and together with the substituted carbon atoms can form an anellated saturated, unsaturated, or aromatic isocyclic or heterocyclic ring having 5-8 ring atoms,

R³⁴, R³⁶, and R⁴⁰ independently of one another denote hydrogen, C₁-C₈-alkyl, phenyl that is unsubstituted or substituted by R³¹ and R³² (except for repeated substitution by R³¹- and R³²-substituted phenyl), C₁-C₈-acyl, C₁-C₈-alkoxycarbonyl, cyano, halogen, carboxyl, C₁-C₈-alkoxy, C₁-C₈-alkylthio, phenylthio, benzylthio, phenoxy, or C₁-C₈-acyloxy,

R³⁵, R³⁷, and R³⁹ independently of one another denote hydrogen, C₁-C₈-alkyl, halogen, C₁-C₈-alkoxy, or C₁-C₈-alkylthio,

R³⁸ denotes hydrogen, C₁-C₈-alkyl, phenyl that is unsubstituted or substituted by R³¹ and R³² (except for repeated substitution by R³¹- and R³²-substituted phenyl), C₁-C₈-acyl, C₁-C₈-thioacyl, halogenocarbonyl, or C₁-C₈-alkoxycarbonyl, and

p represents one of the numbers 0 or 1,

wherein, in addition,

the pairs of substituents R³⁴ and R³⁵, R³⁶ and R³⁷, and R³⁹ and R⁴⁰ independently of one another can denote double-bonded oxygen, sulfur, or R³⁸-substituted nitrogen, and

where, in addition, the substituent pairs R³⁵ and R³⁶, and R³⁸ and R³⁹ independently of one another can form a double bond, and

where, in addition, the substituent pairs R³⁴ and R³⁷, and R³⁸ and R³⁹ independently of one another can form 3- to 5-membered alkylene, in which 1 or 2 carbon atoms can be replaced by oxygen, sulfur, or R³⁸-substituted nitrogen, and

where, in addition, R⁴⁰ can also have the meaning hydrazino, C₁-C₈-alkylhydrazino, or phenylhydrazino.

In addition the co-catalysts used can be, for example, compounds of the formula

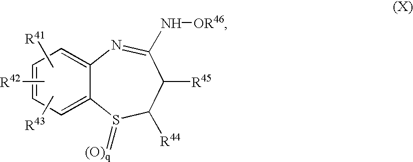

where

R⁴¹ and R⁴² independently of one another denote hydrogen, cyano, halogen, carboxyl, alkoxycarboxyl, alkyl, aryl, alkoxy, aryloxy, or acyl, preferably hydrogen, methyl, ethyl, propyl, or isopropyl,

R⁴³ represents hydrogen, alkyl, or chlorine (preferably hydrogen) and in addition with an adjacently ring-substituted radical R⁴¹ or R⁴² and together with the substituted carbon atoms can form an anellated saturated, unsaturated, or aromatic isocyclic or heterocyclic ring having 5 to 8 ring atoms,

R⁴⁴ and R⁴⁵ independently of one another denote hydrogen, alkyl, aryl, halogen, alkoxy, aryloxy, acyl, or acyloxy (preferably hydrogen, methyl, ethyl, propyl, or isopropyl) or together with the substituted carbon atoms can form a saturated or unsaturated, isocyclic or heterocyclic ring having 5 to 8 ring atoms,

R⁴⁶ denotes hydrogen, alkyl, aryl, or alkyl- or aryl-substituted silyl (preferably hydrogen), and

q can have the value 0 or 1.

In addition, the co-catalysts used can be, for example, compounds of the formula

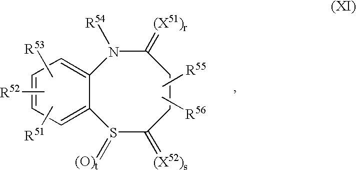

where

R⁵¹ and R⁵² independently of one another denote hydrogen, hydroxyl, amino, cyano, halogen, nitro, alkylsulfonyl, phenylsulfonyl, alkylsulfoxyl, phenylsulfoxyl, tosyl, mercapto, carboxyl, halogenocarbonyl, carboxyamide, alkoxycarbonyl, thiocarboxyamide, alkyl, aryl, heteroaryl, alkoxy, aryloxy, heteroaryloxy, acyloxy, alkylthio, arylthio, heteroarylthio, acylthio, acyl, thioacyl, or acylamino,

R⁵³ represents hydrogen or chlorine and in addition with one of the radicals R⁵¹ or R⁵² and together with the substituted carbon atoms can form an anellated saturated, unsaturated, or aromatic isocyclic or heterocyclic ring having 5 to 8 ring atoms,

R⁵⁴ denotes hydrogen, alkyl, aryl, heteroaryl, acyl, thioacyl, halogenocarbonyl, or alkoxycarbonyl,

X⁵¹ and X⁵² independently of one another represent double-bonded oxygen, sulfur, or R⁵⁷-substituted nitrogen, where R⁵⁷ has the scope of the meanings of R⁵⁴ except hydrogen,

r, s, and t independently of one another can have the value 0 or 1, and

R⁵⁵ and R⁵⁶ independently of one another can be at one or two of the carbon atoms situated between the S atom and the N atom in the 8-membered ring, provided that these carbon atoms are not occupied by X⁵¹ or X⁵², and have the scope of meanings of R⁵¹ and R⁵², where in the case of adjacent ring substitution, with the substituted carbon atoms a saturated, unsaturated, or aromatic isocyclic or heterocyclic ring having 5 to 8 ring atoms can also be formed and, where, in addition, R⁵⁵ and R⁵⁶ together can also denote double-bonded oxygen or sulfur.

Preferably, the co-catalysts used are compounds that contain a seven-membered N- and S-containing heterocycle.

In particular, preferably the co-catalysts used are compounds of the formula (I) where

R¹, R², R³, R⁴, and Y represent hydrogen,

X¹ represents ═O,

X² and X³ independently of one another in each case denote

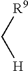

where

R⁹ represents hydrogen, methyl, ethyl, propyl, or isopropyl, and

m denotes 0.

Also, preferably, the co-catalysts used are compounds of the formula (VI) where

R¹, R², R³, and R⁴ are identical or different and represent hydrogen, methyl, ethyl, propyl, or isopropyl,

R⁷ and R⁸ denote hydrogen,

Y represents hydrogen,

X¹ represents ═O,

m denotes the number 0, and

A denotes the anellation of a saturated isocyclic ring having 6 carbon atoms.

In addition, preferably, the co-catalysts used are compounds of the formula (VIII), where

R²¹, R²², R²⁶, and R²⁸ are identical or different and represent hydrogen, methyl, ethyl, propyl, or isopropyl,

R²³ represents hydrogen,

R²⁴ denotes methylthio, ethylthio, propylthio, or isopropylthio,

m and o has the value 0,

n has the value 1, and

R²⁵ and R²⁷ together with the substituted carbon atoms form a saturated isocyclic ring having 6 ring atoms.

It is also possible to use in the inventive process the co-catalysts in combination with other elements or compounds that are not described as co-catalysts.

The co-catalysts can be used not only individually but also in a mixture of a plurality of them.

The amounts of co-catalyst used can vary within broad limits. Amounts less than 0.0001% by weight are less advantageous, since then the co-catalytic activity decreases. Amounts even of 5% by weight or more of co-catalyst can be used, but these high amounts generally offer no advantages, but they may cause disadvantages during work-up. The co-catalysts to be used inventively can therefore be used, for example, in an amount of 0.0001 to 1.0% by weight, preferably 0.0005 to 0.5% by weight, particularly preferably 0.001 to 0.1% by weight, in each case based on the amount of the m-xylene used.

The molar ratio of Friedel-Crafts catalyst(s) or precursors thereof and co-catalyst(s) can be varied within broad limits in the inventive process. A suitable molar ratio, for example, of Friedel-Crafts catalysts or precursors thereof to co-catalyst is 100:1 to 1:50, preferably 75:1 to 1:10, particularly preferably 50:1 to 1:2.

The inventive process is expediently carried out in the liquid phase. If appropriate, the process can be carried out in dilution with an inert solvent.

Suitable solvents are those which are not attacked by chlorine under the conditions of a nuclear chlorination and are known for this purpose to those skilled in the art, such as, for example, methylene chloride, chloroform, carbon tetrachloride, and acetic acid. Preferably no solvent is employed.

The amount of chlorine is preferably selected such that a degree of chlorination of not significantly higher than 1 results. For example, an amount of 0.7 to 1.1 mol of chlorine is used, preferably 0.8 to 1 mol of chlorine, based on the amount of m-xylene used.

The nuclear chlorination to be carried out according to the invention can in principle be carried out at temperatures from the solidification point to the boiling point of the reaction mixture. Generally, the reaction temperature is −30 to 120° C., preferably −10 to 100° C., particularly preferably 0 to 70° C.

The reaction pressure can be atmospheric, reduced, or elevated and is not critical in principle. Because of the inexpensive procedure, atmospheric pressure is preferred. Elevated pressure can be indicated, for example, if the procedure is to be carried out above the boiling point of a low-boiling solvent. In this case, for example, the procedure can be carried out below the inherent pressure of the reaction mixture established by itself.

The water content of the reaction mixture is not generally critical. It is preferred not to dry all starting materials specially, but to use them at the low water content at which they usually occur in chemical engineering. However, it is possible to dry individual or all substances of the reaction mixture specially. Usually, the water content of the starting materials should not be above the saturation limits of the respective starting materials. Water contents in the chlorination mixture are, according to the invention, preferably up to 250 ppm, particularly preferably up to 150 ppm, very particularly preferably up to 100 ppm.

For carrying out the inventive process in practice, the sequence of the addition of the individual components to the reaction mixture is arbitrary. In this case the process may be carried out either continuously or batchwise. For example, m-xylene is charged at the desired reaction temperature, Friedel-Crafts catalyst and co-catalyst are added and the chlorine is added until the desired degree of chlorination is reached. The chlorination mixture can then be worked up directly by distillation. The catalyst components remain behind in the bottom phase.

The inventive process permits the nuclear chlorination of m-xylene having an increased proportion of 4-chloro-1,3-dimethylbenzene with very low amounts of Friedel-Crafts catalysts and co-catalysts being required. Since the process is preferably carried out without solvent, simple work-up is possible by direct distillation of the product mixture.

Using the inventive process, it is possible to achieve, for example, ratios of 4-chloro-1,3-dimethylbenzene to 2-chloro-1,3-dimethylbenzene of 8.05:1, which are thus even higher than the isomer ratios previously obtained using zeolite catalysts.

The examples hereinafter are intended to illustrate the inventive process but without restricting it. The invention, which is set forth in the foregoing disclosure, is not to be limited either in spirit or scope by these examples. Those skilled in the art will readily understand that known variations of the conditions of the following procedures can be used. Unless otherwise noted, all temperatures are degrees Celsius and all percentages are percentages by weight.

### EXAMPLES

### Example 1

100 parts by weight of m-xylene were charged into a blackened chlorination beaker. 152 ppm of FeCl₃ and 45 ppm of a co-catalyst of the formula

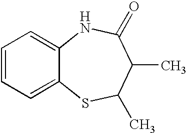

were then added. At 50° C., over the course of 5 h, 95 mol % of chlorine (based on m-xylene) were introduced at a uniform rate. Gas-chromatographic analysis of the reaction mixture found 6.13% m-xylene, 80.11% 4-chloro-1,3-dimethylbenzene, 12.39% 2-chloro-1,3-dimethylbenzene, 1.31% dichlorinated m-xylenes, and 0.06% unknown products.

The ratio of 4-chloro-1,3-dimethylbenzene to 2-chloro-1,3-dimethylbenzene was thus 6.47:1.

### Example 2

The method of Example 1 was repeated, but instead of the co-catalyst used there, 35 ppm of a co-catalyst of the formula

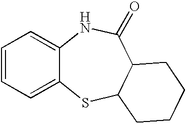

were added. Then, at 50° C., over the course of 5 h, 95 mol % of chlorine (based on m-xylene) were added at a uniform rate. Gas-chromatographic analysis of the reaction mixture found 6.21% m-xylene, 78.43% 4-chloro-1,3-dimethylbenzene, 12.92% 2-chloro-1,3-dimethylbenzene, 2.37% dichlorinated m-xylenes, and 0.07% unknown products. The ratio of 4-chloro-1,3-dimethylbenzene to 2-chloro-1,3-dimethylbenzene was 6.07:1.

### Example 3

The process of Example 1 was repeated, but instead of the co-catalyst used there, 40 ppm of a co-catalyst of the formula

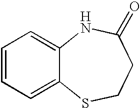

were used and instead of FeCl₃, 250 ppm of SbCl₃ were used. At 40° C., over the course of 6 h, 95 mol % of chlorine (based on m-xylene) were added at a uniform rate. Gas-chromatographic analysis of the reaction mixture found 6.87% m-xylene, 78.50% 4-chloro-1,3-dimethylbenzene, 13.34% 2-chloro-1,3-dimethylbenzene, 1.20% dichlorinated m-xylenes, and 0.09% unknown products, corresponding to a ratio of 4-chloro-1,3-dimethylbenzene to 2-chloro-1,3-dimethylbenzene of 5.88:1.

### Example 4

The process of Example 1 was repeated, but instead of the co-catalyst used there, 56 ppm of a co-catalyst of the formula

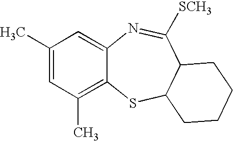

were used. Then, at 50° C., 95 mol % of chlorine (based on m-xylene) were introduced at a uniform rate over the course of 5 h. Gas-chromatographic analysis of the reaction mixture found 7.75% m-xylene, 80.31% 4-chloro-1,3-dimethylbenzene, 9.98% 2-chloro-1,3-dimethylbenzene, 1.86% dichlorinated m-xylenes, and 0.10% unknown products. The ratio of 4-chloro-1,3-dimethylbenzene to 2-chloro-1,3-dimethylbenzene was thus 8.05:1.

### Example 5

The process of Example 1 was repeated, but instead of the co-catalyst used there, 41 ppm of a co-catalyst of the formula

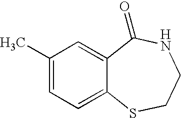

were used. Then, at 50° C., 95 mol % of chlorine (based on m-xylene) were introduced at a uniform rate over the course of 5 h. Gas-chromatographic analysis of the reaction mixture found 5.82% m-xylene, 81.31% 4-chloro-1,3-dimethylbenzene, 10.71% 2-chloro-1,3-dimethylbenzene, 2.04% dichlorinated m-xylenes, and 0.12% unknown products. The ratio of 4-chloro-1,3-dimethylbenzene to 2-chloro-1,3-dimethylbenzene was thus 7.59:1.

### Example 6

The process of Example 1 was repeated, but instead of the co-catalyst used there, 47 ppm of a co-catalyst of the formula

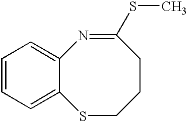

were used. Then, at 50° C., 95 mol % of chlorine (based on m-xylene) were introduced at a uniform rate over the course of 5 h. Gas-chromatographic analysis of the reaction mixture found 7.37% m-xylene, 73.15% 4-chloro-1,3-dimethylbenzene, 17.67% 2-chloro-1,3-dimethylbenzene, 1.40% dichlorinated m-xylenes, and 0.41% unknown products. The ratio of 4-chloro-1,3-dimethylbenzene to 2-chloro-1,3-dimethylbenzene was thus 4.14:1.

### Example 7

The process of Example 1 was repeated, but instead of the co-catalyst used there, 40 ppm of a co-catalyst of the formula

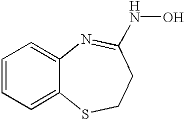

were used. Then, at 50° C., 95 mol % of chlorine (based on m-xylene) were introduced at a uniform rate over the course of 5 h. Gas-chromatographic analysis of the reaction mixture found 5.39% m-xylene, 80.57% 4-chloro-1,3-dimethylbenzene, 12.49% 2-chloro-1,3-dimethylbenzene, 1.48% dichlorinated m-xylenes, and 0.07% unknown products. The ratio of 4-chloro-1,3-dimethylbenzene to 2-chloro-1,3-dimethylbenzene was thus 6.45:1.

### Example 8

The process of Example 1 was repeated, but instead of the co-catalyst used there, 42 ppm of a co-catalyst of the formula

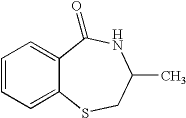

were used. Then, at 50° C., 95 mol % of chlorine (based on m-xylene) were introduced at a uniform rate over the course of 5 h. Gas-chromatographic analysis of the reaction mixture found 7.32% m-xylene, 77.54% 4-chloro-1,3-dimethylbenzene, 12.22% 2-chloro-1,3-dimethylbenzene, 2.75% dichlorinated m-xylenes, and 0.17% unknown products. The ratio of 4-chloro-1,3-dimethylbenzene to 2-chloro-1,3-dimethylbenzene was thus 6.35:1.

### Example 9

The process of Example 1 was repeated, but instead of the co-catalyst used there, 42 ppm of a co-catalyst of the formula

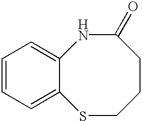

were used. Then, at 50° C., 95 mol % of chlorine (based on m-xylene) were introduced at a uniform rate over the course of 5 h. Gas-chromatographic analysis of the reaction mixture found 8.65% m-xylene, 70.70% 4-chloro-1,3-dimethylbenzene, 15.91% 2-chloro-1,3-dimethylbenzene, 4.36% dichlorinated m-xylenes, and 0.38% unknown products. The ratio of 4-chloro-1,3-dimethylbenzene to 2-chloro-1,3-dimethylbenzene was thus 4.44:1.

### Example 10

The process of Example 1 was repeated, but instead of the co-catalyst used there, 60 ppm of a co-catalyst of the formula

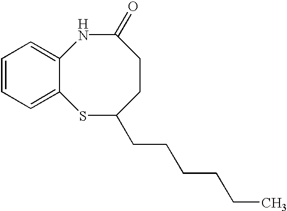

were used. Then, at 50° C., 95 mol % of chlorine (based on m-xylene) were introduced at a uniform rate over the course of 5 h. Gas-chromatographic analysis of the reaction mixture found 6.06% m-xylene, 77.59% 4-chloro-1,3-dimethylbenzene, 14.97% 2-chloro-1,3-dimethylbenzene, 1.13% dichlorinated m-xylenes, and 0.25% unknown products. The ratio of 4-chloro-1,3-dimethylbenzene to 2-chloro-1,3-dimethylbenzene was thus 5.18:1.

What is claimed is:

1. A process for the nuclear chlorination of m-xylene comprising chlorinating m-xylene with elemental chlorine in the presence of Friedel-Crafts catalysts and a benzo-fused thiazepine or thiazocine co-catalyst.

2. A process according to claim 1 wherein the co-catalysts are benzothiazepines of the formulas 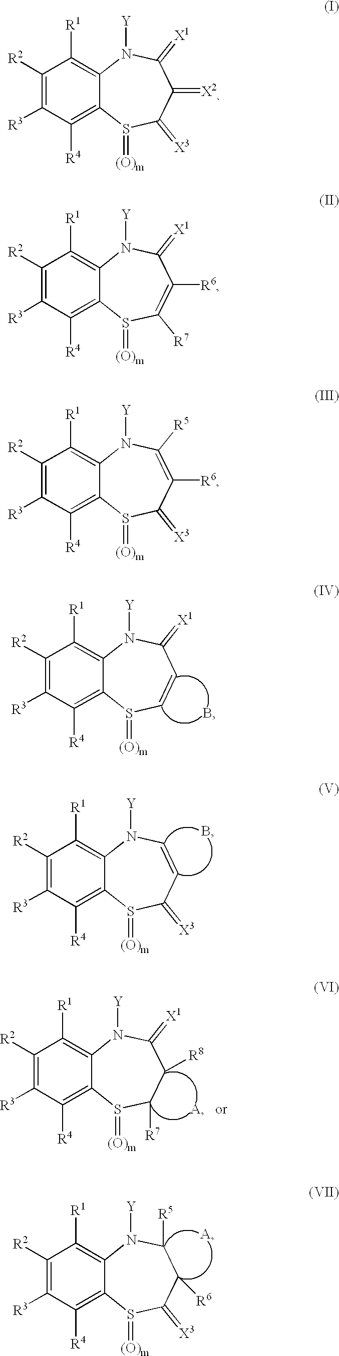
wherein
R¹, R², R³, and R⁴ are identical or different and represent hydrogen, hydroxyl, amino, cyano, halogen, nitro, nitroso, sulfonyl, sulfoxyl, tosyl, mercapto, carboxyl, carboxyamide, carbalkoxy, dithiocarboxyl, thiocarboxyamide, dithiocarbalkoxy, or unsubstituted or substituted alkyl, aryl, heteroaryl, alkoxy, aryloxy, heteroaryloxy, acyloxy, alkylthio, arylthio, heteroarylthio, acylthio, acyl, thioacyl, or acylamino and, in addition, can together form among one another one or more saturated or unsaturated, unsubstituted or substituted isocyclic or heterocyclic carbon rings having up to 8 carbon atoms,
R⁵, R⁶, R⁷, and R⁸ are identical or different and represent hydrogen, hydroxyl, amino, cyano, halogen, nitro, nitroso, sulfonyl, sulfoxyl, tosyl, mercapto, carboxyl, carboxyamide, carbalkoxy, dithiocarboxyl, thiocarboxyamide, dithiocarbalkoxy, or unsubstituted or substituted alkyl, aryl, heteroaryl, alkoxy, aryloxy, heteroaryloxy, acyloxy, alkylthio, arylthio, heteroarylthio, acylthio, acyl, thioacyl, or acylamino,
Y denotes hydrogen, unsubstituted or substituted alkyl, aryl, heteroaryl, acyl, thioacyl, acyloxy, arylamino, or acylamino,
X¹, X², or X³ independently of one another each denotes one of the following groups: 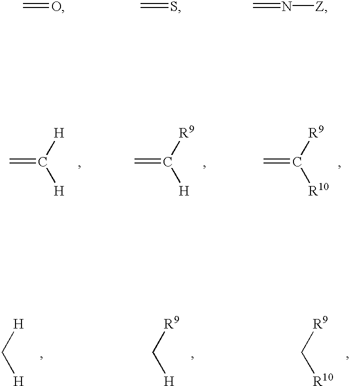
where
R⁹ and R¹⁰ are identical or different and have the meanings of R⁵ to R⁸, and
Z denotes unsubstituted or substituted alkyl, aryl, heteroaryl, acyl, thioacyl, acyloxy, arylamino, or acylamino,
A denotes the anellation of an unsubstituted or substituted saturated isocyclic or heterocyclic ring having up to 8 carbon atoms,
B denotes the anellation of an unsubstituted or substituted unsaturated isocyclic or heterocyclic ring having up to 8 carbon atoms, and
m denotes 0 or 1.

3. A process according to claim 1 wherein the co-catalysts are compounds of the formula 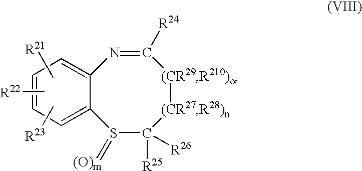
wherein
R²¹ and R²² independently of one another denote hydrogen, hydroxyl, amino, cyano, halogen, nitro, carboxyl, halogenocarbonyl, carboxyamide, alkoxycarbonyl, alkyl, aryl, alkoxy, aryloxy, acyloxy, alkylthio, arylthio, acylthio, acyl, thioacyl, or acylamino,
R²³ represents hydrogen or chlorine and, in addition with an adjacently ring-substituted radical R²¹ or R²² and together with the substituted carbon atoms, can form an anellated saturated, unsaturated, or aromatic isocyclic or heterocyclic ring having 5 to 8 ring atoms,
R²⁴ denotes hydrogen, alkyl, aryl, halogen, alkylthio, arylthio, alkoxy, aryloxy, amino, hydrazino, alkylhydrazino, or phenylhydrazino,
m, n, and o independently of one another can have the value 0 or 1, but n and o cannot simultaneously have the value 0,
R²⁵, R²⁷, and R²⁹ independently of one another denote hydrogen, alkyl, alkoxy, phenyl, acyloxy, cyano, halogen, carboxyl, alkoxycarbonyl, phenoxy, or acyl, where R²⁵ and R²⁷ or R²⁷ and R²⁹, together with the substituted carbon atoms, can form a saturated, unsaturated, or aromatic isocyclic or heterocyclic ring having 5 to 8 ring atoms,
R^{26,} R²⁸, and R²¹⁰ independently of one another denote hydrogen, alkyl, or halogen, where R²⁶ and R²⁸ or R²⁸ and R²¹⁰ can together form a double bond, where, in addition, R²⁵ and R²⁶ can together designate double-bonded oxygen, sulfur, or R²¹¹-substituted nitrogen, where R²¹¹ denotes alkyl, aryl, acyl, alkylamino, or arylamino.

4. A process according to claim 1 wherein the co-catalysts are compounds of the formula 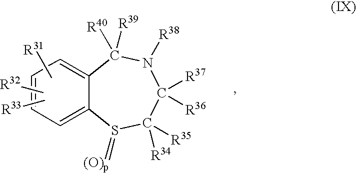
wherein
R³¹ and R³² independently of one another denote hydrogen, hydroxyl, amino, cyano, halogen, nitro, C₁-C₈-alkyl, phenyl that is unsubstituted or substituted by R³¹ and R³² (except for repeated substitution by R³¹- and R³²-substituted phenyl), C₁-C₈-alkoxy, phenoxy, C₁-C₈-acyloxy, C₁-C₈-acyl, or C₁-C₈-alkoxycarbonyl,
R³³ represents hydrogen or chlorine and furthermore with one of the radicals R³¹ or R³² and together with the substituted carbon atoms can form an anellated saturated, unsaturated, or aromatic isocyclic or heterocyclic ring having 5 to 8 ring atoms,
R³⁴, R³⁶, and R⁴⁰ independently of one another denote hydrogen, C₁-C₈-alkyl, phenyl that is unsubstituted or substituted by R³¹ and R³² (except for repeated substitution by R³¹- and R³²-substituted phenyl), C₁-C₈-acyl, C₁-C₈-alkoxycarbonyl, cyano, halogen, carboxyl, C₁-C₈-alkoxy, C₁-C₈-alkylthio, phenylthio, benzylthio, phenoxy, or C₁-C₈-acyloxy,
R³⁵, R³⁷, and R³⁹ independently of one another denote hydrogen, C₁-C₈-alkyl, alkyl, halogen, C₁-C₈-alkoxy, or C₁-C₈-alkylthio,
R³⁸ denotes hydrogen, C₁-C₈-alkyl, phenyl that is unsubstituted or substituted by R³¹ and R³² (except for repeated substitution by R³¹- and R³²-substituted phenyl), C₁-C₈-acyl, C₁-C₈-thioacyl, halogenocarbonyl, or C₁-C₈-alkoxycarbonyl, and
p represents one of the numbers 0 or 1,
wherein, in addition,
the pairs of substituents R³⁴ and R³⁵, R³⁶ and R³⁷, and R³⁹ and R⁴⁰ independently of one another can denote double-bonded oxygen, sulfur, or R³⁸-substituted nitrogen, and
where, in addition, the substituent pairs R³⁵ and R³⁶, and R³⁸ and R³⁹ independently of one another can form a double bond, and
where, in addition, the substituent pairs R³⁴ and R³⁷, and R³⁸ and R³⁹ independently of one another can form 3- to 5-membered alkylene, in which 1 or 2 carbon atoms can be replaced by oxygen, sulfur, or R³⁸-substituted nitrogen, and
where, in addition, R⁴⁰ can also have the meaning hydrazino, C₁-C₈-alkylhydrazino, or phenylhydrazino.

5. A process according to claim 1 wherein the co-catalysts are compounds of the formula 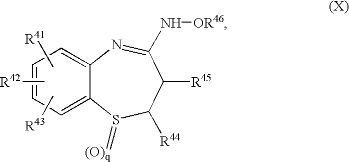
wherein
R⁴¹ and R⁴² independently of one another denote hydrogen, cyano, halogen, carboxyl, alkoxycarboxyl, alkyl, aryl, alkoxy, aryloxy, or acyl,
R⁴³ represents hydrogen, alkyl, or chlorine and in addition with an adjacently ring-substituted radical R⁴¹ or R⁴² and together with the substituted carbon atoms can form an anellated saturated, unsaturated, or aromatic isocyclic or heterocyclic ring having 5 to 8 ring atoms,
R⁴⁴ and R⁴⁵ independently of one another denote hydrogen, alkyl, aryl, halogen, alkoxy, aryloxy, acyl, or acyloxy or together with the substituted carbon atoms can form a saturated or unsaturated, isocyclic or heterocyclic ring having 5 to 8 ring atoms,
R⁴⁶ denotes hydrogen, alkyl, aryl, or alkyl- or aryl-substituted silyl, and
q is 0 or 1.

6. A process according to claim 1 wherein the co-catalysts are compounds of the formula 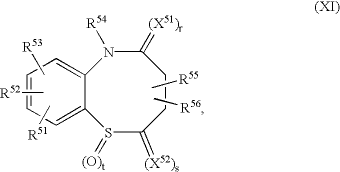
wherein
R⁵¹ and R⁵² independently of one another denote hydrogen, hydroxyl, amino, cyano, halogen, nitro, alkylsulfonyl, phenylsulfonyl, alkylsulfoxyl, phenylsulfoxyl, tosyl, mercapto, carboxyl, halogenocarbonyl, carboxyamide, alkoxycarbonyl, thiocarboxyamide, alkyl, aryl, heteroaryl, alkoxy, aryloxy, heteroaryloxy, acyloxy, alkylthio, arylthio, heteroarylthio, acylthio, acyl, thioacyl, or acylamino,
R⁵³ represents hydrogen or chlorine and in addition with one of the radicals R⁵¹ or R⁵² and together with the substituted carbon atoms can form an anellated saturated, unsaturated, or aromatic isocyclic or heterocyclic ring having 5 to 8 ring atoms,
R⁵⁴ denotes hydrogen, alkyl, aryl, heteroaryl, acyl, thioacyl, halogenocarbonyl, or alkoxycarbonyl,
X⁵¹ and X⁵² independently of one another represent double-bonded oxygen, sulfur, or R⁵⁷-substituted nitrogen, where R⁵⁷ denotes alkyl, aryl, heteroaryl, acyl, thioacyl, halogenocarbonyl, or alkoxycarbonyl,
r, s, and t independently of one another can have the value 0 or 1, and
R⁵⁵ and R⁵⁶ independently of one another can be at one or two of the carbon atoms situated between the S atom and the N atom in the 8-membered ring, provided that these carbon atoms are not occupied by X⁵¹ or X⁵², and have the scope of meanings of R⁵¹ and R⁵², where in the case of adjacent ring substitution, with the substituted carbon atoms a saturated, unsaturated, or aromatic isocyclic or heterocyclic ring having 5 to 8 ring atoms can also be formed and, where, in addition, R⁵⁵ and R⁵⁶ together can also denote double-bonded oxygen or sulfur.

7. A process according to claim 1 wherein the co-catalysts are compounds of the formula (I) 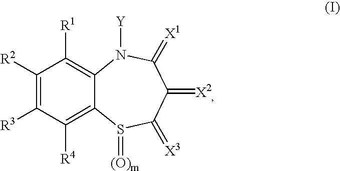
wherein
R¹, R², R³, R⁴, and Y represent hydrogen,
X¹ represents ═O,
X² and X³ independently of one another in each case denote 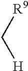
where R⁹ represents hydrogen, methyl, ethyl, propyl, or isopropyl, and
m denotes the number 0.

8. A process according to claim 1 wherein the co-catalysts are compounds of the formula (VI), 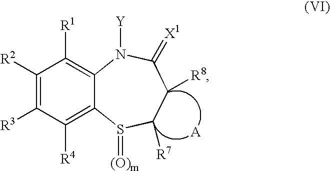
wherein
R¹, R², R³, and R⁴ are identical or different and represent hydrogen, methyl, ethyl, propyl, or isopropyl,
R⁷ and R⁸ denote hydrogen,
Y represents hydrogen,
X¹ represents ═O,
m denotes the number 0, and
A denotes the anellation of a saturated isocyclic ring having 6 carbon atoms.

9. A process according to claim 1 wherein the co-catalysts are compounds of the formula (VIII) 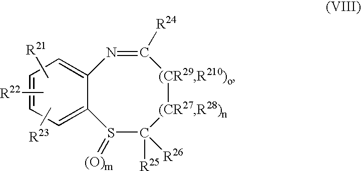
wherein
R²¹, R²², R²⁶, and R²⁷ are identical or different and represent hydrogen, methyl, ethyl, propyl, or isopropyl,
R²³ represents hydrogen,
R²⁴ denotes methylthio, ethylthio, propylthio, or isopropylthio,
m and o have the value 0,
n has the value 1, and
R²⁵ and R²⁷ together with the substituted carbon atoms form a saturated isocyclic ring having 6 ring atoms.

10. A process according to claim 1 wherein the co-catalyst is used in an amount of 0.0001% by weight to 1.0% by weight, based on the amount of the m-xylene.
